(19) United States
(12) Patent Application Publication
(10) Pub. No.: US 2024/0277804 A1
(43) Pub. Date: Aug. 22, 2024

(54) TRIPLE COMBINATION THERAPY

(71) Applicant: Immutep S.A.S., SAINT-AUBIN (FR)

(72) Inventor: Frederic TRIEBEL, SAINT-AUBIN (FR)

(73) Assignee: Immutep S.A.S., SAINT-AUBIN (FR)

(21) Appl. No.: 18/570,846

(22) PCT Filed: Jun. 20, 2022
(86) PCT No.: PCT/EP2022/066760

§ 371 Date: Dec. 15, 2023

(30) Foreign Application Priority Data
Jun. 18, 2021 (GB) 2108718.4

(51) Int. Cl. A61K 38/17; A61K 31/282; A61K 31/519; A61K 39/395; A61P 35/00

(52) U.S. Cl. CPC A61K 38/1774; A61K 31/282; A61K 31/519; A61K 39/3955; A61P 35/00

(57) ABSTRACT

Use of a LAG-3 protein or derivative thereof as part of a combination therapy for the treatment of cancer is described. In particular. (a) a LAG-3 protein, or a derivative thereof that is able to bind to MIC class II molecules. (b) a programmed cell death protein-1 (PD-1) pathway inhibitor, and (c) a chemotherapy agent, for use in preventing, treating, or ameliorating a cancer in a subject is described. Combined preparations, and pharmaceutical compositions, comprising: (a) a LAG-3 protein, or derivative thereof that is able to bind to MHC class II molecules: (b) a programmed cell death protein-1 (PD-1) pathway inhibitor; and (c) a chemotherapy agent, are also described. Use of the combined preparations and compositions as medicaments, in particular for the prevention, treatment, or amelioration of cancer are also described, as are methods for the prevention, treatment, or amelioration of cancer.

### FIELD OF THE INVENTION

This invention relates to the use of a LAG-3 protein or derivative thereof as part of a combination therapy for the treatment of cancer.

### BACKGROUND OF THE INVENTION

Over the past decade, PD-1 and CTLA-4 immune checkpoint inhibitors such as OPDIVO (nivolumab), KEYTRUDA (pembrolizumab) and YERVOY (ipilimumab) have become the standard of care therapies for many forms of cancer, however unfortunately, many patients still fail to respond to these modern medicines. In some cases, these new medicines are combined with chemotherapy (chemo-IO) to improve response rates, although this can lead to undesirable toxic effects. In other cases, combinations of immune checkpoint inhibitors (IO-IO) are used, but this can also lead to undesirable toxic effects.

To improve patient outcomes, significant work has been undertaken to investigate other immune checkpoints, such as LAG-3, TIM-3, VISTA, CD47, IDO, and TIGIT. LAG-3 in particular has emerged as a promising checkpoint and several companies are developing new inhibitors that target this checkpoint. The aim of a LAG-3 inhibitor, as with the currently approved PD-1 and CTLA-4 inhibitors, is to block the down-regulation of the immune system i.e. take the “brakes off” the body's immune processes. Significant work has also been undertaken to explore combinations of PD-1 and CTLA-4 immune checkpoint inhibitors with other approved or experimental therapies.

Another type of active immunotherapy being investigated are the antigen presenting cell (APC) activators. APC activators bind to antigen presenting cells such as dendritic cells, monocytes and macrophages via MHC II molecules. This activates the APCs causing them to become professional antigen presenting cells, thereby presenting antigen to the adaptive immune system. This leads to activation and proliferation of CD4+(helper) and CD8+(cytotoxic) T cells. Thus, the aim of APC activators is to “push the gas” on the body's immune system.

Eftilagimod alpha (IMP321 or efti), a soluble dimeric recombinant form of LAG-3, is a first-in-class APC activator under clinical development. By stimulating dendritic cells and other APCs through MHC class II molecules, IMP321 induces a powerful anti-cancer T cell response. IMP321 is described in WO 2009/044273, which also describes the use of IMP321 alone and in combination with a chemotherapy agent for the treatment of cancer. In addition, WO 2016/110593 describes the use of IMP321 in combination with a PD-1 pathway inhibitor for the treatment of cancer and infectious disease.

There remains a need in the art for improved cancer therapies and treatment regimens leading to better outcomes for patients. This is especially so for cancers where the prognosis for patients undertaking treatment with current approved medicines is poor and/or where current medicines are poorly tolerated.

### SUMMARY OF THE INVENTION

In one embodiment, the invention relates to (a) a LAG-3 protein, or a derivative thereof that is able to bind to MHC class II molecules, (b) a programmed cell death protein-1 (PD-1) pathway inhibitor, and (c) a chemotherapy agent, for use in preventing, treating, or ameliorating a cancer in a subject.

In another embodiment, the invention relates to the use of (a) a LAG-3 protein, or a derivative thereof that is able to bind to MHC class II molecules, (b) a programmed cell death protein-1 (PD-1) pathway inhibitor, and (c) a chemotherapy agent, in the manufacture of a medicament for the prevention, treatment, or amelioration of a cancer in a subject.

In yet another embodiment, the invention relates to the use of (a) a LAG-3 protein, or a derivative thereof that is able to bind to MHC class II molecules, (b) a programmed cell death protein-1 (PD-1) pathway inhibitor, and (c) a chemotherapy agent, for the prevention, treatment, or amelioration of a cancer in a subject.

In a further embodiment, the invention provides a method of preventing, treating, or ameliorating a cancer in a subject, the method comprising administering to the subject in need of such prevention, treatment, or amelioration (a) a LAG-3 protein, or a derivative thereof that is able to bind to MHC class II molecules, (b) a programmed cell death protein-1 (PD-1) pathway inhibitor, and (c) a chemotherapy agent.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered simultaneously or sequentially with a programmed cell death protein-1 (PD-1) pathway inhibitor and a chemotherapy agent.

In one embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered simultaneously or sequentially with a programmed cell death protein-1 (PD-1) pathway inhibitor and a chemotherapy agent.

In another embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, wherein the LAG-3 protein or derivative thereof is administered simultaneously or sequentially with a programmed cell death protein-1 (PD-1) pathway inhibitor and a chemotherapy agent.

In yet another embodiment, the invention provides a combined preparation, comprising:
(a) a LAG-3 protein, or derivative thereof that is able to bind to MHC class II molecules,
(b) a programmed cell death protein-1 (PD-1) pathway inhibitor, and
(c) a chemotherapy agent.

In a further embodiment, the invention provides a pharmaceutical composition, comprising:
(a) a LAG-3 protein, or derivative thereof that is able to bind to MHC class II molecules,
(b) a programmed cell death protein-1 (PD-1) pathway inhibitor,
(c) a chemotherapy agent, and
(d) a pharmaceutically acceptable carrier, excipient, or diluent.

### BRIEF DESCRIPTION OF THE DRAWINGS

FIG. 1 illustrates the amino acid sequence of mature human LAG-3 protein. The four extracellular Ig superfamily domains are at amino acid residues: 1-149 (D1); 150-239 (D2); 240-330 (D3); and 331-412 (D4). The amino acid sequence of the extra-loop structure of the D1 domain of human LAG-3 protein is shown underlined in bold.

FIG. 2 illustrates shrinkage of a target tumour lesion of a NSCLC patient measured by computed tomography (CT) (A: August 2021 and B: May 2022). The lesion shrunk from 22.62 mm in diameter to “evaluable but not measurable”. The lesion is shown with a dashed circle.

FIG. 3 illustrates shrinkage of another target tumour lesion of the NSCLC patient measured by computed tomography (CT) (A: August 2021 and B: May 2022). The lesion shrunk from 35.92 mm to 25.70 mm (in diameter) and is shown with a dashed circle.

### DETAILED DESCRIPTION OF THE INVENTION

### Triple Combination Therapy

In one embodiment, the invention relates to (a) a LAG-3 protein, or a derivative thereof that is able to bind to MHC class II molecules, (b) a programmed cell death protein-1 (PD-1) pathway inhibitor, and (c) a chemotherapy agent, for use in preventing, treating, or ameliorating a cancer in a subject.

In another embodiment, the invention relates to the use of (a) a LAG-3 protein, or a derivative thereof that is able to bind to MHC class II molecules, (b) a programmed cell death protein-1 (PD-1) pathway inhibitor, and (c) a chemotherapy agent, in the manufacture of a medicament for the prevention, treatment, or amelioration of a cancer in a subject.

In yet another embodiment, the invention relates to the use of (a) a LAG-3 protein, or a derivative thereof that is able to bind to MHC class II molecules, (b) a programmed cell death protein-1 (PD-1) pathway inhibitor, and (c) a chemotherapy agent, for the prevention, treatment, or amelioration of a cancer in a subject.

In a further embodiment, the invention provides a method of preventing, treating, or ameliorating a cancer in a subject, the method comprising administering to the subject in need of such prevention, treatment, or amelioration (a) a LAG-3 protein, or a derivative thereof that is able to bind to MHC class II molecules, (b) a programmed cell death protein-1 (PD-1) pathway inhibitor, and (c) a chemotherapy agent.

In yet a further embodiment, the invention relates to a LAG-3 protein, or a derivative thereof that is able to bind to MHC class II molecules, for use in preventing, treating, or ameliorating a cancer in a subject, wherein the LAG-3 protein or derivative thereof is to be administered simultaneously or sequentially with a programmed cell death protein-1 (PD-1) pathway inhibitor and a chemotherapy agent.

In one embodiment, the invention relates to the use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered simultaneously or sequentially with a programmed cell death protein-1 (PD-1) pathway inhibitor and a chemotherapy agent.

In another embodiment, the invention provides a method of preventing, treating, or ameliorating a cancer in a subject, the method comprising administering to the subject in need of such prevention, treatment, or amelioration a LAG-3 protein, or a derivative thereof that is able to bind to MHC class II molecules, wherein the LAG-3 protein or derivative thereof is administered simultaneously or sequentially with a programmed cell death protein-1 (PD-1) pathway inhibitor and a chemotherapy agent.

In yet another embodiment, the invention provides a combined preparation, comprising:
(a) a LAG-3 protein, or derivative thereof that is able to bind to MHC class II molecules,
(b) a programmed cell death protein-1 (PD-1) pathway inhibitor, and
(c) a chemotherapy agent.

In a further embodiment, the invention provides a pharmaceutical composition, comprising:
(a) a LAG-3 protein, or derivative thereof that is able to bind to MHC class II molecules,
(b) a programmed cell death protein-1 (PD-1) pathway inhibitor,
(c) a chemotherapy agent, and
(d) a pharmaceutically acceptable carrier, excipient, or diluent.

Exemplary cancers that may be treated according to the invention include, but are not limited to, breast cancer, skin cancer, lung cancer (for example NSCLC or SCLC), ovarian cancer, renal cancer (for example renal cell carcinoma), colon cancer, colorectal cancer, gastric cancer, esophageal cancer, pancreatic cancer, bladder cancer, urothelial cancer, liver cancer, melanoma (for example, metastatic malignant melanoma), prostate cancer (for example hormone refractory prostate adenocarcinoma), head and neck cancer (for example, head and neck squamous cell carcinoma), cervical cancer, endometrial cancer, uterine cancer, thyroid cancer, glioblastoma, glioma, leukemia, lymphoma (for example, a B cell lymphoma or Hodgkin lymphoma), adrenal gland cancer, AIDS-associated cancer, alveolar soft part sarcoma, astrocytic tumor, bone cancer, brain and spinal cord cancer, metastatic brain tumor, carotid body tumor, chondrosarcoma, chordoma, cutaneous benign fibrous histiocytoma, desmoplastic small round cell tumor, ependymoma, Ewing's tumor, extraskeletal myxoid chondrosarcoma, fibrogenesis imperfecta ossium, fibrous dysplasia of the bone, gallbladder or bile duct cancer, gestational trophoblastic disease, germ cell tumor, haematological malignancy, hepatocellular carcinoma, islet cell tumor, Kaposi's sarcoma, kidney cancer, lipoma/benign lipomatous tumor, liposarcoma/malignant lipomatous tumor, medulloblastoma, meningioma, Merkel cell carcinoma, multiple endocrine neoplasia, multiple myeloma, myelodysplasia syndrome, neuroblastoma, neuroendocrine tumor, papillary thyroid carcinoma, parathyroid tumor, pediatric cancer, peripheral nerve sheath tumor, phaeochromocytoma, pituitary tumor, prostate cancer, posterior uveal melanoma, rare hematologic disorder, rhabdoid tumor, rhabdomysarcoma, sarcoma, soft-tissue sarcoma, squamous cell cancer, synovial sarcoma, mesothelioma, cutaneous squamous cell carcinoma, testicular cancer, thymic carcinoma, thymoma, and thyroid metastatic cancer.

Exemplary cancers that may be treated according to the invention include, but are not limited to, rectal cancer, anal cancer, small intestine cancer, gastrointestinal stromal tumours.

In one embodiment, the cancer is a lung cancer. In another embodiment, the lung cancer is non-small cell lung cancer (NSCLC). In yet another embodiment, the lung cancer is small cell lung cancer (SCLC).

NSCLC includes: (a) non-squamous cell carcinoma (adenocarcinoma, large cell, and undifferentiated carcinoma), (b) squamous cell carcinoma, and (c) non-small cell carcinoma not otherwise specified.

In one embodiment, the NSCLC is non-squamous NSCLC. In another embodiment, the NSCLC is squamous NSCLC.

In one embodiment, the cancer is a gastrointestinal cancer. Suitably, the gastrointestinal cancer is anal cancer, bile duct cancer, colon cancer, rectal cancer, esophageal cancer, gallbladder cancer, gastrointestinal stromal tumours, liver cancer, pancreatic cancer, small intestine cancer, or gastric cancer.

In one embodiment, the cancer is a head and neck cancer. In another embodiment, the head and neck cancer is head and neck squamous cell carcinoma (HNSCC).

In one embodiment, the cancer is a breast cancer. Suitably, the breast cancer is an adenocarcinoma of the breast.

According to embodiments of the invention, the cancer may have progressed to metastatic disease.

In one particular embodiment, the cancer is metastatic NSCLC. In an embodiment, the metastatic NSCLC is non-squamous NSCLC. In another embodiment, the metastatic NSCLC is squamous NSCLC.

Suitably, patients with metastatic NSCLC are treated with the triple combination therapy as a 1^{st} line therapy. Alternatively, patients with metastatic NSCLC are treated with the triple combination therapy as a 2^{nd} line therapy.

PD-L1 expression status is a well-known predictive marker for response to PD-1 pathway inhibitors including in NSCLC and HNSCC. For example, PD-L1 expression is typically reported in three groups for NSCLC: <1%, 1-49% and ≥50% (Tumour Proportion Score or TPS) and in HNSCC: <1, 1-19 and ≥20 (Combined Positive Score or CPS). Patients with a high PD-L1 status are typically more responsive to PD-1 pathway inhibitors, whereas those with a low PD-L1 status are overall significantly less responsive.

In an embodiment, the subject has NSCLC and a low PD-L1 expression status (e.g. <50%, 1-49%, or <1%) and would otherwise be less likely to respond to therapy with a PD-1 pathway inhibitor, if not for the triple combination therapy of the invention.

In an embodiment, the subject has NSCLC and is treated without regard to their PD-L1 expression status.

In another embodiment, the subject has NSCLC and a PD-L1 expression status of <50%.

In yet another embodiment, the subject has NSCLC and a PD-L1 expression status of 1-49%.

In a further embodiment, the subject has NSCLC and a PD-L1 expression status of <1%.

In yet a further embodiment, the subject has NSCLC and a PD-L1 expression status of ≥1%.

In an embodiment, the subject has NSCLC and a PD-L1 expression status of ≥50%.

Suitably, the NSCLC is metastatic NSCLC.

### LAG-3 Protein and Derivatives

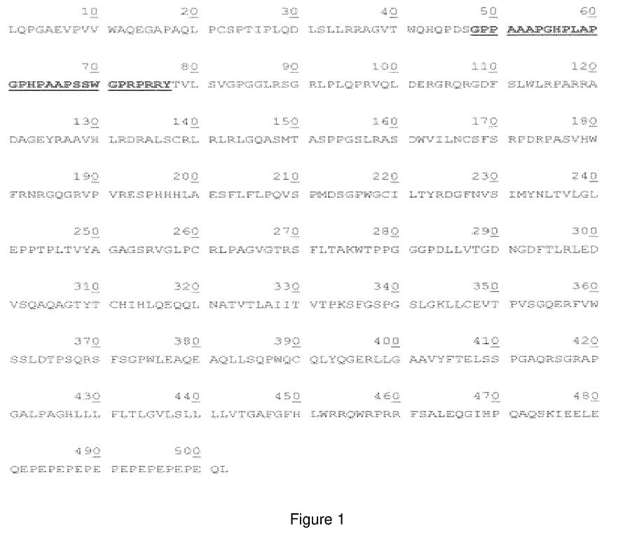

According to embodiments of the invention, the LAG-3 protein may be an isolated natural or recombinant LAG-3 protein. The LAG-3 protein may comprise an amino acid sequence of LAG-3 protein from any suitable species, such as a primate or murine LAG-3 protein, but preferably a human LAG-3 protein. The amino acid sequence of human and murine LAG-3 protein is provided in FIG. 1 of Huard et al (Proc. Natl. Acad. Sci. USA, 11: 5744-5749, 1997). The sequence of human LAG-3 protein is repeated in FIG. 1 herein (SEQ ID NO: 1). The amino acid sequences of the four extracellular Ig superfamily domains (D1, D2, D3, and D4) of human LAG-3 are also identified in FIG. 1 of Huard et al., at amino acid residues: 1-149 (D1); 150-239 (D2); 240-330 (D3); and 331-412 (D4).

Derivatives of LAG-3 protein include soluble fragments, variants, or mutants of LAG-3 protein that are able to bind to MHC class II molecules. Several derivatives of LAG-3 protein are known that are able to bind to MHC class II molecules. Many examples of such derivatives are described in Huard et al (Proc. Natl. Acad. Sci. USA, 11: 5744-5749, 1997). This document describes characterization of the MHC class II binding site on LAG-3 protein. Methods for making mutants of LAG-3 are described, as well as a quantitative cellular adhesion assay for determining the ability of LAG-3 mutants to bind to class II-positive Daudi cells. Binding of several different mutants of LAG-3 to MHC class II molecules was determined. Some mutations were able to reduce class II binding, while other mutations increased the affinity of LAG-3 for class II molecules. Many of the residues essential for binding of LAG-3 to MHC class II proteins are clustered at the base of a large 30 amino acid extra-loop structure in the LAG-3 D1 domain. The amino acid sequence of the extra-loop structure of the D1 domain of human LAG-3 protein is GPPAAAPGHPLAPGPHPAAPSSWGPRPRRY (SEQ ID NO:2). The amino acid sequence of the extra-loop structure of the D1 domain of human LAG-3 protein is shown underlined in bold in FIG. 1.

In an embodiment of the invention, the derivative of LAG-3 protein comprises the 30 amino acid extra-loop sequence of the human LAG-3 D1 domain, or a variant of such sequence with one or more amino acid substitutions (e.g. a conservative amino acid substitution). The variant may comprise an amino acid sequence that has at least 70%, 80%, 90%, or 95% amino acid identity with the 30 amino acid extra-loop sequence of the human LAG-3 D1 domain.

The derivative of LAG-3 protein may comprise an amino acid sequence of domain D1, domain D1 and optionally D2, or domains D1 and D2, of LAG-3 protein, preferably human LAG-3 protein.

The derivative of LAG-3 protein may comprise an amino acid sequence that has at least 70%, 80%, 90%, or 95% amino acid identity with domain D1, domain D1 and optionally D2, or domains D1 and D2, of LAG-3 protein, preferably human LAG-3 protein.

The derivative of LAG-3 protein may comprise an amino acid sequence of domains D1, D2, and D3, domains D1, D2, D3 and optionally D4, or domains D1, D2, D3 and D4, of LAG-3 protein, preferably human LAG-3 protein.

The derivative of LAG-3 protein may comprise an amino acid sequence that has at least 70%, 80%, 90%, or 95% amino acid identity with domains D1, D2 and D3, domains D1, D2, D3 and optionally D4, or with domains D1, D2, D3 and D4, of LAG-3 protein, preferably human LAG-3.

Sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

The derivative of LAG-3 protein may be fused to Immunoglobulin Fc amino acid sequence, preferably human lgG1 Fc amino acid sequence, optionally by a linker amino acid sequence. The ability of a derivative of LAG-3 protein to bind to MHC class II molecules may be determined using a quantitative cellular adhesion assay as described in Huard et al (Proc. Natl. Acad. Sci. USA, 11: 5744-5749, 1997). The affinity of a derivative of LAG-3 protein for MHC class II molecules may be at least 20%, 30%, 40%, 50%, 60%, 70%, 80%, 90%, or 100% of the affinity of human LAG-3 protein for MHC class II molecules.

Preferably, the affinity of a derivative of LAG-3 protein for MHC class II molecules is at least 50%, 60%, 70%, 80%, 90%, 95%, 99%, or 100% of the affinity of human LAG-3 protein for MHC class II molecules.

Examples of suitable derivatives of LAG-3 protein that are able to bind to MHC class II molecules include derivatives comprising:
amino acid residues 23 to 448 of the human LAG-3 sequence;
amino acid sequence of domains D1 and D2 of LAG-3;
amino acid sequence of domains D1 and D2 of LAG-3 with an amino acid substitution at one or more of the following positions: position 30 where ASP is substituted with ALA; position 56 where HIS is substituted with ALA; position 73 where ARG is substituted with GLU; position 75 where ARG is substituted with ALA or GLU; position 76 where ARG is substituted with GLU; or position 103 where ARG is substituted with ALA; and a recombinant soluble human LAG-3lg fusion protein (IMP321)-a 160-kDa dimer produced in Chinese hamster ovary cells transfected with a plasmid encoding for the extracellular domain of hLAG-3 fused to the human IgG1 Fc. The sequence of IMP321 is given in SEQ ID NO: 17 of US 2011/0008331.

In an embodiment, the subject is a mammal, preferably a human.

According to the invention, the LAG-3 protein or derivative thereof is administered in a therapeutically effective amount. A “therapeutically effective amount” refers to an amount of the active ingredient sufficient to have a therapeutic effect upon administration. Effective amounts of the active ingredient may vary, for example, with the particular disease or diseases being treated, the severity of the disease, the duration of the treatment, and characteristics of the patient (e.g. sex, age, height and weight).

In an embodiment, the LAG-3 protein or derivative thereof is administered at a dose which is a molar equivalent of about 0.1 mg to about 60 mg, about 6 mg to about 60 mg, about 10 mg to about 50 mg, about 20 mg to about 40 mg, about 25 mg to about 35 mg, or about 30 mg of the LAG-3 derivative LAG-3lg fusion protein IMP321.

In another embodiment, the LAG-3 protein or derivative thereof is administered at a dose which is a molar equivalent of about 25 mg, about 26 mg, about 27 mg, about 28 mg, about 29 mg, about 30 mg, about 31 mg, about 32 mg, about 33 mg, about 34 mg, or about 35 mg of the LAG-3 derivative LAG-3lg fusion protein IMP321.

Suitably, the LAG-3 protein or derivative thereof is administered at a dose which is a molar equivalent of about 30 mg of the LAG-3 derivative LAG-3lg fusion protein IMP321.

In yet another embodiment, the LAG-3 protein or derivative thereof is administered at a dose which is a molar equivalent from about 25 mg to about 60 mg, such as about 25 mg, about 30 mg, about 35 mg, about 40 mg, about 45 mg, about 50 mg, about 55 mg, or about 60 mg, of the LAG-3 derivative LAG-3lg fusion protein IMP321.

In one embodiment, the LAG-3 protein or derivative thereof is IMP321 and is administered at a dose of about 0.1 mg to about 60 mg, about 6 mg to about 60 mg, about 10 mg to about 50 mg, about 20 mg to about 40 mg, about 25 mg to about 35 mg, or about 30 mg.

In another embodiment, the IMP321 is administered at a dose of about 25 mg, about 26 mg, about 27 mg, about 28 mg, about 29 mg, about 30 mg, about 31 mg, about 32 mg, about 33 mg, about 34 mg, or about 35 mg.

Suitably, IMP321 is administered at a dose of about 30 mg.

In other embodiments, IMP321 is administered at a dose from about 25 mg to about 60 mg, such as about 25 mg, about 30 mg, about 35 mg, about 40 mg, about 45 mg, about 50 mg, about 55 mg, or about 60 mg.

Doses of 6-30 mg per subcutaneous (s.c.) injection of IMP321 have been shown, thus far, to be safe and provide an acceptable systemic exposure based on the results of pharmacokinetics data obtained in cancer patients. A blood concentration of IMP321 superior to 1 ng/ml for at least 24 hours after s.c. injection is obtained in patients injected with IMP321 doses of more than 6 mg. No dose limiting toxicity has been observed to date.

In an embodiment, the LAG-3 protein or derivative thereof is administered about once every week to the subject. In another embodiment, the LAG-3 protein or derivative thereof is administered about once every two weeks to the subject. In yet another embodiment, the LAG-3 protein or derivative thereof is administered about once every three weeks to the subject. In a further embodiment, the LAG-3 protein or derivative thereof is administered about once every four weeks to the subject. In yet a further embodiment, the LAG-3 protein or derivative thereof is administered about once every month to the subject. As will be appreciated by those of skill in the art, the precise treatment regimen may vary and be adapted according to the particular cancer being treated and characteristics of the patient.

In one embodiment, the LAG-3 protein or derivative thereof is present in the absence of any additional antigen added to the pharmaceutical composition, combined preparation, or medicament.

### PD-1 Pathway Inhibitor

The PD-1 pathway inhibitor is an agent that inhibits binding of PD-1 to PD-L1 and/or PD-L2. In particular, the agent may inhibit binding of human PD-1 to human PD-L1 and/or human PD-L2. The agent may inhibit binding of PD-1 to PD-L1 and/or PD-L2 by at least 50%, 60%, 70%, 80%, or 90%. Suitable assays for determining binding of PD-1 to PD-L1 or PD-L2, by Surface Plasmon Resonance (SPR) analysis, or flow cytometry analysis, are described in Ghiotto et al (Int. Immunol. August 2010; 22(8): 651-660). The agent may inhibit binding of PD-1 to PD-L1 and/or PD-L2, for example, by binding to PD-1, to PD-L1, or to PD-L2.

The agent may be an antibody, suitably a monoclonal antibody, such as a human or humanized monoclonal antibody. The agent may be a fragment or derivative of an antibody that retains ability to inhibit binding of PD-1 to PD-L1 and/or PD-L2.

Exemplary PD-1 pathway inhibtors include, but are not limited to, pembrolizumab, nivolumab, cemiplimab, spartalizumab, camrelizumab, sintilimab, tislelizumab, toripalimab, dostarlimab, atezolizumab, avelumab, and durvalumab, or a fragment or derivative thereof that retains ability to inhibit binding of PD-1 to PD-L1 and/or PD-L2.

Suitably, the PD-1 pathway inhibitor is an anti-PD-1 antibody selected from the group consisting of pembrolizumab, nivolumab, cemiplimab, spartalizumab, camrelizumab, sintilimab, tislelizumab, toripalimab, and dostarlimab, or a fragment or derivative thereof that retains ability to inhibit binding of PD-1 to PD-L1 and/or PD-L2.

Suitably, the PD-1 pathway inhibitor is an anti-PD-L1 antibody selected from the group consisting of atezolizumab, avelumab, and durvalumab, or a fragment or derivative thereof that retains ability to inhibit binding of PD-L1 to PD-1.

Suitably, the PD-1 pathway inhibitor is pembrolizumab.

Suitably, the PD-1 pathway inhibitor is nivolumab.

Suitably, the PD-1 pathway inhibitor is cemiplimab.

Suitably, the PD-1 pathway inhibitor is spartalizumab.

Suitably, the PD-1 pathway inhibitor is camrelizumab.

Suitably, the PD-1 pathway inhibitor is sintilimab.

Suitably, the PD-1 pathway inhibitor is tislelizumab.

Suitably, the PD-1 pathway inhibitor is toripalimab.

Suitably, the PD-1 pathway inhibitor is dostarlimab.

Suitably, the PD-1 pathway inhibitor is atezolizumab.

Suitably, the PD-1 pathway inhibitor is avelumab.

Suitably, the PD-1 pathway inhibitor is durvalumab.

Other exemplary PD-1 pathway inhibitors include JTX-4014, INCMGA00012, AMP-224, AMP-514, KN035, CK-301, AUNP12, CA-170 and BMS-986189.

The dose of the PD-1 pathway inhibitor will depend on the particular PD-1 pathway inhibitor being used. In general, a typically prescribed dose of a PD-1 pathway inhibitor for a human subject may be 0.1 to 10 mg/kg, for example 0.1 to 1 mg/kg, or 1 to 10 mg/kg. The term “typically prescribed dose” is used herein to include a dose which is the same as the dose, or within the dosage range, that is safe and therapeutically effective for administration to a subject (suitably a human subject).

In an embodiment, the PD-1 pathway inhibitor is administered about once every week to the subject. In another embodiment, the PD-1 pathway inhibitor is administered about once every two weeks to the subject. In yet another embodiment, the PD-1 pathway inhibitor is administered about once every three weeks to the subject. In a further embodiment, the PD-1 pathway inhibitor is administered about once every four weeks to the subject. In yet a further embodiment, the PD-1 pathway inhibitor is administered about once every month to the subject. In an embodiment, the PD-1 pathway inhibitor is administered about once every five weeks to the subject. In another embodiment, the PD-1 pathway inhibitor is administered about once every six weeks to the subject. In yet another embodiment, the PD-1 pathway inhibitor is administered about once every seven weeks to the subject. In yet another embodiment, the PD-1 pathway inhibitor is administered about once every eight weeks to the subject. In a further embodiment, the PD-1 pathway inhibitor is administered about once every two months to the subject.

As will be appreciated by those of skill in the art, the precise treatment regimen may vary and be adapted according to the particular cancer being treated and characteristics of the patient.

Examples of typically prescribed human doses of known PD-1 pathway inhibitors include:
Pembrolizumab: 200 mg every three weeks or 400 mg every six weeks.
Nivolumab: 240 mg every two weeks, 360 mg every 3 weeks, or 480 mg every 4 weeks
Avelumab: 800 mg every two weeks (or maximum dose 10 mg/kg if weight <80 kg).

In some embodiments, the PD-1 pathway inhibitor is administered parenterally (including by subcutaneous, intravenous, or intramuscular injection) or orally.

Suitably, the PD-1 pathway inhibitor is administered intravenously.

### Chemotherapy Agent

Suitable chemotherapy agents include, but are not limited to, alkylating agents, plant alkaloids, antitumor antibiotics, antimetabolites, topoisomerase inhibitors, and miscellaneous antineoplastics, and mixtures thereof.

Suitably, the chemotherapy agent is an alkylating agent. Exemplary alkylating agents include mustard gas derivatives such mechlorethamine, cyclophosphamide, chlorambucil, melphalan, and ifosfamide; ethylenimines such as thiotepa and hexamethylmelamine; alkylsulfonates such as busulfan; hydrazines and triazines such as altretamine, procarbazine, dacarbazine and temozolomide; nitrosureas such as carmustine, lomustine and streptozocin; and platinum chemotherapy agents such as carboplatin, cisplatin, oxaliplatin, nedaplatin, triplatin tetranitrate, phenanthriplatin, picoplatin, and satraplatin.

Suitably, the chemotherapy agent is a plant alkaloid. Exemplary plant alkaloids include vinca alkaloids such as vincristine, vinblastine and vinorelbine; taxanes such as paclitaxel, nab-paclitaxel, docetaxel, cabazitaxel, larotaxel, milataxel, ortataxel, taxoprexin, opaxio, tesetaxel, and BMS-184476; podophyllotoxins such as etoposide and tenisopide; and camptothecan analogs such as irinotecan and topotecan.

Suitably, the chemotherapy agent is an antitumor antibiotic. Exemplary antitumor antibiotics include anthracyclines such as doxorubicin, daunorubicin, epirubicin, mitoxantrone, and idarubicin; chromomycins such as dactinomycin and plicamycin; and miscellaneous antitumor antibiotics such as mitomycin and bleomycin.

Suitably, the chemotherapy agent is an antimetabolite. Exemplary antimetabolites include folic acid antagonists such as methotrexate and pemetrexed; pyrimidine antagonists such as 5-fluorouracil, tegafur, carmofur, doxifluridine, floxuridine, cytarabine, capecitabine and gemcitabine; purine antagonists such as 6-mercaptopurine and 6-thioguanine; and adenosine deaminase inhibitors such as cladribine, fludarabine, nelarabine and pentostatin.

Suitably, the chemotherapy agent is a topoisomerase inhibitor. Exemplary topoisomerase inhibitors include topoisomerase I inhibitors such as irinotecan and topotecan; and topoisomerase II inhibitors such as amsacrine, etoposide, etoposide phosphate and teniposide.

Suitably, the chemotherapy agent is a miscellaneous antineoplastic. Exemplary miscellaneous antineoplastics include ribonucleotide reductase inhibitors such as hydroxyurea; adrenocortical steroid inhibitors such as mitotane; enzymes such as asparaginase and pegaspargase; antimicrotubule agents such as estramustine; and retinoids such bexarotene, isotretinoin and tretinoin.

Suitably, the chemotherapy agent is a combination of two or more chemotherapy agents.

Suitably, the chemotherapy agent is a combination of two chemotherapy agents.

In one embodiment, the chemotherapy agent is a combination of pemetrexed and platinum chemotherapy such as carboplatin, cisplatin, or oxaliplatin.

In another embodiment, the chemotherapy agent is a combination of pemetrexed and carboplatin.

In yet another embodiment, the chemotherapy agent is carboplatin and a taxane such as paclitaxel or nab-paclitaxel.

In a further embodiment, the chemotherapy agent is carboplatin and paclitaxel or nab-paclitaxel.

The chemotherapy agent is administered in a therapeutically effective amount. A therapeutically effective amount refers to an amount of the chemotherapy agent sufficient to have a therapeutic effect upon administration. Effective amounts of the chemotherapy agent will vary with the chemotherapy agent selected, the particular disease or diseases being treated, the severity of the disease, the duration of the treatment, and characteristics of the patient (e.g. sex, age, height and weight).

In some embodiments, the chemotherapy agent is administered parenterally (including by subcutaneous, intravenous, or intramuscular injection) or orally.

Suitably, the chemotherapy is administered intravenously.

In an embodiment, the chemotherapy agent is administered about once every week to the subject. In another embodiment, the chemotherapy agent is administered about once every two weeks to the subject. In yet another embodiment, the chemotherapy agent is administered about once every three weeks to the subject. In a further embodiment, the chemotherapy agent is administered about once every four weeks to the subject. In yet a further embodiment, the chemotherapy agent is administered about once every month to the subject.

In an embodiment, the LAG-3 protein or derivative thereof is administered simultaneously or sequentially with the PD-1 pathway inhibitor and the chemotherapy agent.

In another embodiment, the LAG-3 protein or derivative thereof is administered sequentially with the PD-1 pathway inhibitor and the chemotherapy agent.

In yet another embodiment, the chemotherapy agent is administered and is followed by sequential administration of the LAG-3 protein or derivative thereof and the PD-1 pathway inhibitor.

### Doses of Components of Triple Combination Therapy

The doses of the components used in the triple combination therapy according to the invention should be chosen to provide a therapeutically effective amount of the components in combination. An “effective amount” of the triple combination therapy may be an amount that results in a reduction of at least one pathological parameter associated with cancer. For example, in some embodiments, an effective amount of the triple combination therapy is an amount that is effective to achieve a reduction of at least about 10%, 20%, 30%, 40%, 50%, 60%, 70%, 80%, or 90%, in the pathological parameter, compared to the expected reduction in the parameter associated with the cancer without the triple combination therapy. For example, the pathological parameter may be tumor growth, or tumor growth rate.

Alternatively, an “effective amount” of the triple combination therapy may be an amount that results in an increase in a clinical benefit associated with cancer treatment. For example, in some embodiments, an “effective amount” of the combination therapy is an amount that is effective to achieve an increase of at least about 10%, 20%, 30%, 40%, 50%, 60%, 70%, 80%, 90%, 100%, 125%, 150%, 175% or 200%, in the clinical benefit, compared to the expected clinical benefit without the triple combination therapy. For example, the clinical benefit may be response rate, progression-free survival, overall survival, disease control rate, depth of response, duration of response, quality of life, or increased sensitization to subsequent treatments.

Alternatively, an “effective amount” of the triple combination therapy may be an amount that results in a change of at least one beneficial parameter relating to cancer treatment. For example, in some embodiments, an “effective amount” of the triple combination therapy is an amount that is effective to achieve a change of at least about 10%, 20%, 30%, 40%, 50%, 60%, 70%, 80% or 90%, in the parameter, compared to the expected change in the parameter relating to cancer treatment without the combination therapy. For example, the parameter may be an increase in the number of circulating tumor antigen-specific CD8⁺ T cells, or a reduction in the number of tumor antigen-specific regulatory T cells, or an increase in the number of activated T cells, in particular activated CD8⁺ T cells, a reduction in the number of exhausted antigen-specific CD8⁺ T cells, or an increase in the number of circulating functional (i.e. non-exhausted) antigen-specific CD8⁺ T cells.

According to the invention, triple combination therapy may be employed to increase the therapeutic effect of the PD-1 pathway inhibitor and/or the chemotherapy agent, compared with (a) the effect of the PD-1 pathway inhibitor and the chemotherapy agent as monotherapies or (b) a combination therapy consisting of the PD-1 pathway inhibitor and the chemotherapy agent.

Triple combination therapy may also be employed to decrease the doses of the individual components in the combination while preventing or further reducing the risk of unwanted or harmful side effects of the individual components.

In an embodiment, the dosage of the PD-1 pathway inhibitor and/or chemotherapy agent is less than a typically prescribed dose for monotherapy with the PD-1 pathway inhibitor or chemotherapy agent, or below a typically prescribed dose for a combination therapy consisting of the PD-1 pathway inhibitor and chemotherapy agent, for example, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5%, of the typically prescribed dose of the PD-1 pathway inhibitor and/or chemotherapy agent.

In another embodiment, the dosage of the PD-1 pathway inhibitor and/or chemotherapy agent is less than a typically prescribed dose for monotherapy with the PD-1 pathway inhibitor or chemotherapy agent, or below a typically prescribed dose for a combination therapy consisting of the PD-1 pathway inhibitor and chemotherapy agent, for example, from about 25% to about 75%, or from about 1% to about 50%, or from about 0.5% to about 25%, of the typically prescribed dose of the PD-1 pathway inhibitor and/or chemotherapy agent.

In yet another embodiment, the dosage of the PD-1 pathway inhibitor and the chemotherapy agent is in accordance with the prescribed standard of care.

Suitably, the course of triple combination therapy takes place over, for example, about 3 months, about 4 months, about 5 months, about 6 months, about 7 months, about 8 months, about 9 months, about 10 months, about 11 months or about 12 months.

Similarly, the course of combination therapy takes place over, for example, about 12 weeks, about 16 weeks, about 20 weeks, about 24 weeks, about 28 weeks, about 32 weeks, about 36 weeks, about 40 weeks, about 44 weeks, about 48 weeks or about 52 weeks.

In one embodiment, the course of triple combination therapy takes place over about 16 weeks. In another embodiment, the course of triple combination therapy takes place over about 24 weeks.

Suitably, after the subject is treated with the triple combination therapy, the subject moves to a maintenance phase.

In an embodiment, the maintenance phase comprises a LAG-3 protein or derivative thereof, a PD-1 pathway inhibitor, and a chemotherapy agent, wherein the chemotherapy agent is a single chemotherapy agent.

Suitably, the maintenance phase takes place over about 16 weeks to about 52 weeks, such as about 20 weeks, about 22 weeks, about 24 weeks, about 26 weeks, about 28 weeks, about 30 weeks, about 32 weeks, about 34 weeks, about 36 weeks, about 38 weeks, or about 40 weeks.

In an embodiment, the maintenance phase takes place over about 4 months to about 12 months, such as about 6 months.

In another embodiment, the subject is treated with the triple combination therapy for up to 24 weeks and then proceeds to a maintenance phase for a total treatment duration of up to 52 weeks.

In one particular embodiment, the total treatment duration including the triple combination therapy and the maintenance phase is up to about 52 weeks.

In another embodiment, the total duration of therapy including the triple combination therapy and maintenance phase is about 12 months, about 15 months, or about 18 months.

Alternatively, after the subject is treated with the triple combination therapy, the subject moves to a chemotherapy-free maintenance phase comprising the LAG-3 protein or derivative thereof and the PD-1 pathway inhibitor.

The chemotherapy-free maintenance phase may be, for example, for about 6 months, about 9 months, about 12 months, about 15 months, about 18 months, about 21 months, or about 24 months.

### Combined Preparations

In one embodiment, the LAG-3 protein or derivative thereof, the PD-1 pathway inhibitor and the chemotherapy agent are packaged separately. That is, in this embodiment, the LAG-3 protein or derivative thereof, the PD-1 pathway inhibitor, and the chemotherapy agent are separate unit dosage forms, which would typically (but not necessarily) be sourced from different suppliers, and then used in the methods of the invention.

In another embodiment, the LAG-3 protein or derivative thereof, the PD-1 pathway inhibitor and the chemotherapy agent are in the form of a combined preparation.

The three components of the “combined preparation” may be present:
(i) in one combined unit dosage form known as a fixed dose combination (FDC), or
(ii) as a first unit dosage form of component (a); a separate, second unit dosage form of component (b); and a separate, third unit dosage form of component (c) and where the three separate dosage forms are packaged together known as a kit-of-parts.

The ratio of the total amounts of the combination components (a), (b) and (c) to be administered in the combined preparation can be varied, for example, in order to cope with the needs of a patient sub-population to be treated, or the needs of the patient, which can be due, for example, to the particular disease, age, sex, or body weight of the patient.

That is, the combined preparation according to the invention may take the form of a pharmaceutical composition comprising the LAG-3 protein or derivative thereof, the PD-1 pathway inhibitor and the chemotherapy agent or, alternatively, as a kit-of-parts comprising the LAG-3 protein or derivative thereof, the PD-1 pathway inhibitor, and the chemotherapy agent as separate components, but packaged together.

Thus, in an embodiment, the invention provide a combined preparation, comprising:
(a) a LAG-3 protein, or derivative thereof that is able to bind to MHC class II molecules,
(b) a PD-1 pathway inhibitor, and
(c) a chemotherapy agent.

The combined preparation may comprise a plurality of doses of the LAG-3 protein or derivative thereof, a plurality of doses of the PD-1 pathway inhibitor, and/or a plurality of doses of the chemotherapy agent.

In another embodiment, one of the three components is packaged separately and the other two components are packaged together as a combined preparation. The two components of the combined preparation may be present as (i) a FDC, or (ii) a kit-of-parts.

In one embodiment, the LAG-3 protein or derivative thereof is packaged separately and the the PD-1 pathway inhibitor and the chemotherapy agent are a combined preparation.

### Pharmaceutical Compositions

The LAG-3 protein or derivative thereof, the PD-1 pathway inhibitor, and the chemotherapy agent are formulated with a pharmaceutically acceptable carrier, excipient, or diluent to provide a pharmaceutical composition. Typically these will be formulated as separate pharmaceutical compositions, although in the case of a fixed dose combination, the LAG-3 protein or derivative thereof, the PD-1 pathway inhibitor and the chemotherapy agent will be formulated together, along with a pharmaceutically acceptable carrier, excipient, or diluent.

The separate pharmaceutical compositions may be packaged together in the form of a kit-of-parts or sourced separately for use in the methods of the invention.

In general, the LAG-3 protein or derivative thereof, the PD-1 pathway inhibitor, and the chemotherapy agent may be administered by known means, in any suitable pharmaceutical composition, by any suitable route.

Suitable pharmaceutical compositions may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate compositions of the invention in a unit dosage form for ease of administration and uniformity of dosage. The term “unit dosage form” as used herein refers to physically discrete units suited as unitary dosages for the individuals to be treated. That is, the compositions are formulated into discrete dosage units each containing a predetermined “unit dosage” quantity of an active agent calculated to produce the desired therapeutic effect in association with the required pharmaceutical carrier, excipient or diluent. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

Preferably, there is at least one beneficial effect from the triple combination therapy, for example, advantageous therapeutic effects (e.g. overall response rate, progression-free survival, overall survival, disease control rate, depth of response or duration of response), fewer side effects, less toxicity, or improved QOL-compared with an effective dosage of one or two of components (a), (b) and (c).

### EXAMPLES

Embodiments of the invention are now described, by way of example only, with reference to the accompanying drawings in which:

### Example 1-Active Immunotherapy IMP321 in Combined Therapy with a PD-1 Pathway Inhibitor and a Chemotherapy Agent (Trial INSIGHT, EudraCT No. 2016-002309-20)

A clinical study was carried out to investigate the safety and efficacy of the active immunotherapy IMP321 in combination with a PD-1 pathway inhibitor and a chemotherapy agent in patients with various solid tumours including NSCLC. It was planned to enrol 20 patients.

In particular, patients with non-squamous 1st line metastatic NSCLC were treated with biweekly IMP321 (30 mg s.c.) in parallel with a standard of care combination of pemetrexed (500 mg/m2), carboplatin (AUC5) and pembrolizumab (200 mg) administered every three weeks, for up to 24 weeks. Thereafter, patients moved to maintenance therapy for a total study duration of up to 52 weeks.

The maintenance therapy generally consisted of IMP321 (30 mg s.c.) administered either every 2 weeks or every 3 weeks in parallel with pemetrexed (500 mg/m2) and pembrolizumab (200 mg) administered every 3 weeks. Maintenance therapy may alternatively consist of therapy with IMP321 (30 mg) and pembrolizumab (200 mg) only (chemotherapy-free).

Patients stayed on the study until disease progression, unacceptable toxicity, completion of the maintenance phase or discontinuation for any other reason. Treatment beyond disease progression is an option in the presence of a clinical benefit.

After the maintenance phase, patients were followed up for 12 months or until disease progression, whichever is earlier.

Additional radiation therapy is permitted. In case of bone metastases, the administration of bisphosphonates is permitted. Irradiation on target lesions is not allowed.

As of the end of May 2022, 11/20 non-squamous 1^{st} line metastatic NSCLC patients had been enrolled into the trial, thus far. In 8 evaluable patients, there were 4 confirmed partial responses, 3 patients with stable disease, and only 1 patient with disease progression (interim disease control rate in evaluable patients: 87.5%).

No additional toxicity was observed from treatment with the triple combination therapy compared to treatment with a combination of pembrolizumab and chemotherapy in historical trials. No adverse events leading to discontinuation from the trial were observed, thus far.

### Single Patient Case Study:

Bipulmonary metastatic lung carcinoma originating from the right lower lobe
Born 1949
ECOG=1
Adenocarcinoma
Thyroid transcription factor (TTF) negative
PD-L1: TPS=0 (IC 0%)
No driver mutations
pT2a, pNO, RO
Malignancy grade G2
Ipsilateral pleural dissemination (pM1a)
Histological confirmation of pulmonary metastasis contralateral (left upper lobe of lung)

The patient with a more limited prognosis was treated with the triple combination therapy and has since moved to a maintenance phase of therapy with a combination of IMP321 and pembrolizumab only. The patient has stable disease and remains under therapy with an ECOG status=1.

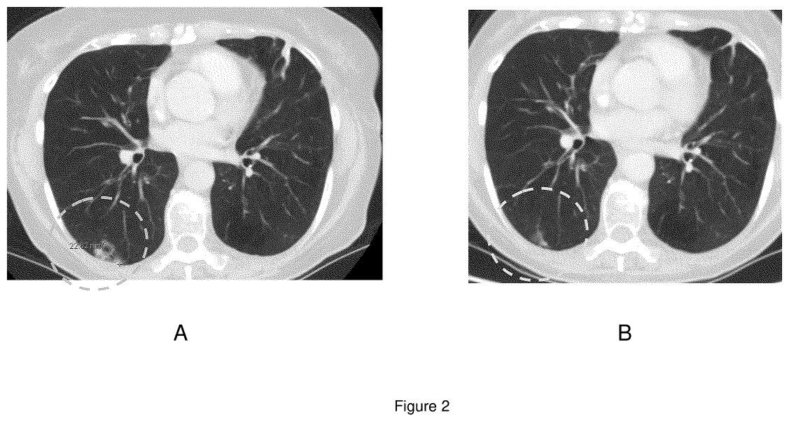

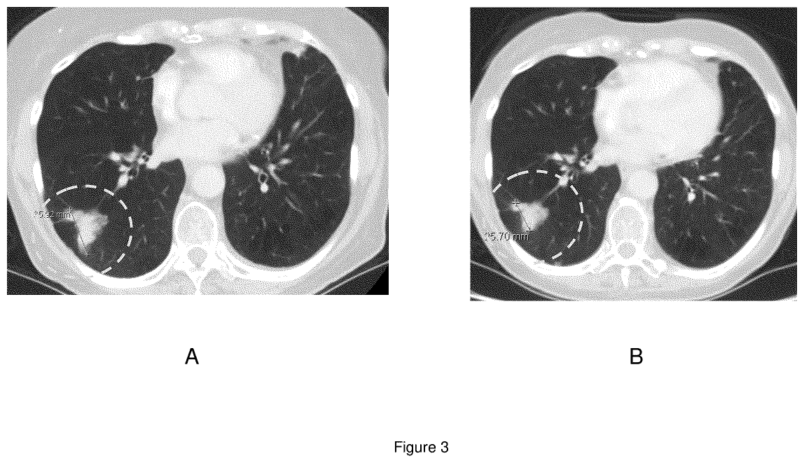

CT scans of the thorax region of the patient have shown shrinkage of target tumour lesions in the course of therapy. An example of the shrinkage of a target lesion is shown in FIG. 2 where the lesion shrunk from 22.62 mm in diameter to “evaluable but not measurable”. Similarly, FIG. 3 illustrates the shrinkage of another target lesion: this one from 35.92 mm in diameter to 25.70 mm.

1. Use of (a) a LAG-3 protein, or a derivative thereof that is able to bind to MHC class II molecules, (b) a programmed cell death protein-1 (PD-1) pathway inhibitor and (c) a chemotherapy agent, in the manufacture of a medicament for the prevention, treatment, or amelioration of a cancer in a subject.

2. Use of a LAG-3 protein, or a derivative thereof that is able to bind to MHC class II molecules, in the manufacture of a medicament for the prevention, treatment, or amelioration of a cancer in a subject, wherein the LAG-3 protein or derivative thereof is to be administered simultaneously or sequentially with a programmed cell death protein-1 (PD-1) pathway inhibitor and a chemotherapy agent.

3. Use according to claim 1 or 2, wherein the derivative of LAG-3 protein comprises an amino acid sequence that has at least 70% amino acid identity with domain D1, and optionally domain D2, of LAG-3 protein, preferably human LAG-3 protein.

4. Use according to any preceeding claim, wherein the derivative of LAG-3 protein comprises an amino acid sequence that has at least 70% amino acid identity with domains D1, D2 and D3, and optionally domain D4, of LAG-3 protein, preferably human LAG-3 protein.

5. Use according to any preceeding claim, wherein the cancer is selected from the group consisting of breast cancer, skin cancer, lung cancer (NSCLC or SCLC), ovarian cancer, renal cancer (for example renal cell carcinoma), colon cancer, rectal cancer, colorectal cancer, anal cancer, small intestine cancer, gastrointestinal stromal tumours, gastric cancer, esophageal cancer, pancreatic cancer, bladder cancer, urothelial cancer, liver cancer, melanoma (for example, metastatic malignant melanoma), prostate cancer (for example hormone refractory prostate adenocarcinoma), head and neck cancer (for example, head and neck squamous cell carcinoma), cervical cancer, endometrial cancer, uterine cancer, thyroid cancer, glioblastoma, glioma, leukemia, lymphoma (for example, a B cell lymphoma or Hodgkin lymphoma), adrenal gland cancer, AIDS-associated cancer, alveolar soft part sarcoma, astrocytic tumor, bone cancer, brain and spinal cord cancer, metastatic brain tumor, carotid body tumor, chondrosarcoma, chordoma, cutaneous benign fibrous histiocytoma, desmoplastic small round cell tumor, ependymoma, Ewing's tumor, extraskeletal myxoid chondrosarcoma, fibrogenesis imperfecta ossium, fibrous dysplasia of the bone, gallbladder or bile duct cancer, gestational trophoblastic disease, germ cell tumor, haematological malignancy, hepatocellular carcinoma, islet cell tumor, Kaposi's sarcoma, kidney cancer, lipoma/benign lipomatous tumor, liposarcoma/malignant lipomatous tumor, medulloblastoma, meningioma, Merkel cell carcinoma, multiple endocrine neoplasia, multiple myeloma, myelodysplasia syndrome, neuroblastoma, neuroendocrine tumor, papillary thyroid carcinoma, parathyroid tumor, pediatric cancer, peripheral nerve sheath tumor, phaeochromocytoma, pituitary tumor, prostate cancer, posterior uveal melanoma, rare hematologic disorder, rhabdoid tumor, rhabdomysarcoma, sarcoma, soft-tissue sarcoma, squamous cell cancer, synovial sarcoma, mesothelioma, cutaneous squamous cell carcinoma, testicular cancer, thymic carcinoma, thymoma, and thyroid metastatic cancer.

6. Use according to any preceeding claim, wherein the cancer is a lung cancer, preferably NSCLC.

7. Use according to any proceeding claim, wherein the PD-1 pathway inhibitor is selected from the group consisting of pembrolizumab, nivolumab, cemiplimab, spartalizumab, camrelizumab, sintilimab, tislelizumab, toripalimab, dostarlimab, atezolizumab, avelumab, and durvalumab.

8. Use according to any proceeding claim, wherein the PD-1 pathway inhibitor is pembrolizumab.

9. Use according to any proceeding claim, wherein the chemotherapy agent is a combination of two or more chemotherapy agents.

10. Use according to any proceeding claim, wherein the LAG-3 derivative is IMP321, the PD-1 pathway inhibitor is pembrolizumab, the chemotherapy agent comprises a combination of pemetrexed and carboplatin, and the cancer is NSCLC, preferably non-squamous NSCLC.

11. Use according to any proceeding claim, wherein the PD-L1 expression level of the subject is <50%.

12. Use according to claim 11, wherein the PD-L1 expression level of the subject is 1-49%.

13. Use according to claim 11, wherein the PD-L1 expression level of the subject is <1%.

14. Use according to any proceeding claim, wherein the subject is a human.


SEQUENCE LISTING (2 sequences)

SEQ ID NO: 1 - protein, 502 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPQAQSKIEELEQEPEPEPEPEPEPEPEPEPEQL

SEQ ID NO: 2 - protein, 30 aa, Homo sapiens
GPPAAAPGHPLAPGPHPAAPSSWGPRPRRY